(19) United States
(12) Patent Application Publication
(10) Pub. No.: US 2007/0224640 A1
(43) Pub. Date: Sep. 27, 2007

(54) Device and Method for Protein Analysis

(75) Inventors: Karin Dahlgren Caldwell, Djursholm (SE); Margaretha Andersson, Bjorklinge (SE); Karin Fromell, Grillby (SE)

Correspondence Address: YOUNG & THOMPSON, 745 SOUTH 23RD STREET, 2ND FLOOR, ARLINGTON, VA, 22202, US

(73) Assignee: AMERSHAM BIOSCIENCES AB, Uppsala (SE)

(21) Appl. No.: 11/578,991

(22) PCT Filed: Apr. 15, 2005
(86) PCT No.: PCT/SE05/00552

§ 371 Date: Jan. 18, 2007

(30) Foreign Application Priority Data
Apr. 20, 2004 (SE) 0401033-6

(51) Int. Cl. G01N 33/68

(52) U.S. Cl. 435/7.1

(57) ABSTRACT

A device for protein analysis or mapping, includes an array of particulate nanoparticles, wherein said nanoparticles are bound, preferably co-immobilized, at specific spots on a planar read-out surface or substrate and are provided with a plurality of capture probes for capturing proteins, preferably glycoproteins. A method in which the device is used for protein profiling, especially glycoprotein profiling, of individual samples with high sensitivity is also disclosed. The device and method do not require any complex sample preparation.

### FIELD OF THE INVENTION

The present invention relates to a device and a method for protein analysis, especially glycoprotein analysis or glycoprotein mapping. The device comprises a read-out surface with spots of particulate nanoparticles with specific protein binding ligands, such as carbohydrate binding lectins for binding of glycoproteins. The lectins are concentrated on the nanoparticles and bind different glycoproteins with high sensitivity.

In the method of the invention a sample is flowed over the spots on the device and binding to lectins or other ligands is detected and analysed. The method and device do not require any complex sample preparation.

### BACKGROUND OF THE INVENTION

The completed map of the human genome clearly indicates that the genetic code contains blue prints of considerably fewer proteins than originally expected. This implies that proteomic research must increase attention to post-translational modification such as glycosylation. Most plasma, membrane and secretor proteins are glycosylated. Identification of glycoprotein glycoforms is becoming increasingly important as more and more diseases are found to correlate with glycan structure alterations [Durand G. et al, Rudd, Rudd science, Jaeken]. Classical methods for glycoprotein profiling are often time-consuming, include elaborate sample preparations, and are not always suitable for “real-world” situations with heterogeneous samples in complex matrixes, such as blood plasma or crude cell extracts.

Protein microarrays are of considerable interest for mapping protein expression, alterations and interactions. Yet, they have turned out to be more difficult to achieve than their counterparts for DNA analysis, largely because proteins are harder to work with than nucleic acids [P Mitchell, Nature]. For instance, there are no methods to amplify protein expression like PCR amplifies mRNA. Instead, detectability must be intensified by increased local concentration of analyte.

Lectins are carbohydrate-binding proteins with one or more specific binding sites per molecule. They have very specific affinities towards an assortment of carbohydrate structures and are known to identify those molecules within a population of proteins that contain their particular binding partners. Lectins are usually specific for only one or two types of monosaccharides and it is crucial how the saccharide is presented on the glycoprotein [Sharon Turner, C Nilsson].

There are several ways to attach proteins to surfaces. One common way is to let the protein nonspecifically adsorb directly to a particle surface such as that presented by polystyrene (PS) latex beads. The problem is that polystyrene surfaces are highly hydrophobic, which frequently leads to denaturation of the adsorbed protein followed by loss of activity. [Fromell Huang Cald, G Yan et al, Norde et al]. This makes direct adsorption of limited applicability as an immobilization method for bioactive proteins.

Known methods and devices using lectins to detect and analyse carbohydrates need to be improved, primarily the sensitivity need to be increased.

### SUMMARY OF THE INVENTION

The present inventors increase the analytical sensitivity in protein analysis by immobilizing the receptor molecule, e.g. lectins, on a small particle, which in turn is attached to a planar, read-out surface with low non-specific uptake of proteins. Nanoparticles constitute convenient platforms for the attachment of bioactive proteins, since protein coated nanoparticles present high concentrations of attachment sites for specific ligands and offer minimal sterical hindrance to binding. Small nanoparticles expose a several-fold higher surface area for modification compared to other commonly used flat surfaces e.g. microtiter plates. The binding activity of proteins is also proven to depend on the curvature of the surface, to which they are attached, where higher curvature leads to increased binding constants.

According to the invention the receptor molecules, preferably lectins, are selectively immobilized to the surface via a tether of the synthetic surfactant Pluronic F108-PDS preadsorbed to the PS surface. Pluronic F108-PDS is an end group activated triblock copolymer with two relatively hydrophilic polyethyleneoxide (PEO) blocks flanking a hydrophobic polypropyleneoxide (PPO) block responsible for the adsorption. The PEO sidechains have been activated by the introduction of a pyridyldisulfide group (PDS), to which thiol-containing molecules such as our specially thiolated lectins, can be covalently attached. The advantage of using the Pluronic F108-PDS as a tether is that it shields the hydrophobic surface and thereby protects the attached proteins from denaturation while at the same time preventing unspecific protein adsorption [Lee, Basinska, Neff, P.].

Thus, the present invention relates to a method for glycoprotein profiling, preferably using lectins as capture probes immobilized on particulate substrates in the nm-range. The nanoparticles present high concentrations of attachment sites for specific ligands and cause minimal steric hindrance to binding.

In a first aspect, the invention relates to a device for protein analysis or mapping, comprising an array of particulate nanoparticles, wherein said nanoparticles are bound, preferably co-immobilized, at specific spots on a planar read-out surface or substrate and are provided with a plurality of capture probes or ligands for capturing proteins. The proteins may be any proteins, such as glycosylated or phosphorylated proteins and the capture probes are suitable ligands binding to glycosylated and phosphorylated proteins, respectively. Preferably the proteins to be analysed are glycosylated proteins and the capture probes are lectins.

In the present invention, a panel of lectins is used for glycoprotein differential mapping in miniaturized high throughput screening devices. These devices are designed to separate proteins with respect to their glycosylation pattern using arrays of lectin coated nanoparticles. The affinity panel will be interpreted using pattern recognition techniques. A schematic view of the nanoparticle microarray platform is seen in FIG. 1.

The array of nanoparticles may be divided into several sections and each section or row, e.g. a row with five spots on the substrate, having different capture probes attached to the nanoparticles. It is also contemplated that each spot may be provided with nanoparticles having a unique capture probe for a separate spot.

Preferably, the nanoparticles are bound to the solid phase by oligomers on the particles and complementary oligomers on the solid phase, for example a dG oligomer on the particles and a dC oligomer on the solid phase. The length of the oligomer is for example a 15-mer but may longer or shorter.

In the examples, the nanoparticles are made of polystyrene but any other polymer may be used, such as a low fluorescent polymer. The particulate nanoparticles may be, for example, spherical.

Preferably, the capture probes are attached to the nanoparticles via a polyethyleneoxide linker. The lectins or other carbohydrate binding ligands may be any lectins such as mannose-specific lectins: Concanavalin A/ConA, type 1 fimbriae, favin, GNL, LOL, LCL, MBP-A, PSL; N-acetylglucoseamine-specific lectins: GSII, WGA; galactose/N-acetylgalactoseamine-specific lectins: jacalin, DBL, ECorL, LBA, MLL, PNA, RCAII, SBA; fucose-specific lectins: LTA, UEA I; sialic acid-specific lectins: lectin from Sambucus nigra (elderberry), etc. The names/abbreviations used for the lectins are conventional.

In a second aspect, the invention relates to a method for protein analysis, comprising the following steps:
a) application of a (preferably labelled, see experimental below) sample to the device described above,
b) binding of proteins to the capture probes,
c) incubation,
d) washing off excess sample, and
e) detection of possibly bound proteins to the capture probes.

The sample may be any sample, such as body fluid or cell extract from an individual
The proteins may be glycosylated or phosphorylated proteins or any other proteins one wishes to analyse. In this way a unique glycoprotein and/or phosphoprotein profile may be obtained for one individual. Preferably the proteins are glycoproteins. If one so desires the glycoproteins may be further analysed after this step by for example mass spectrometry for identification.

### BRIEF DESCRIPTION OF THE DRAWINGS

FIG. 1. Schematic description of the nanoparticle microarray platform.

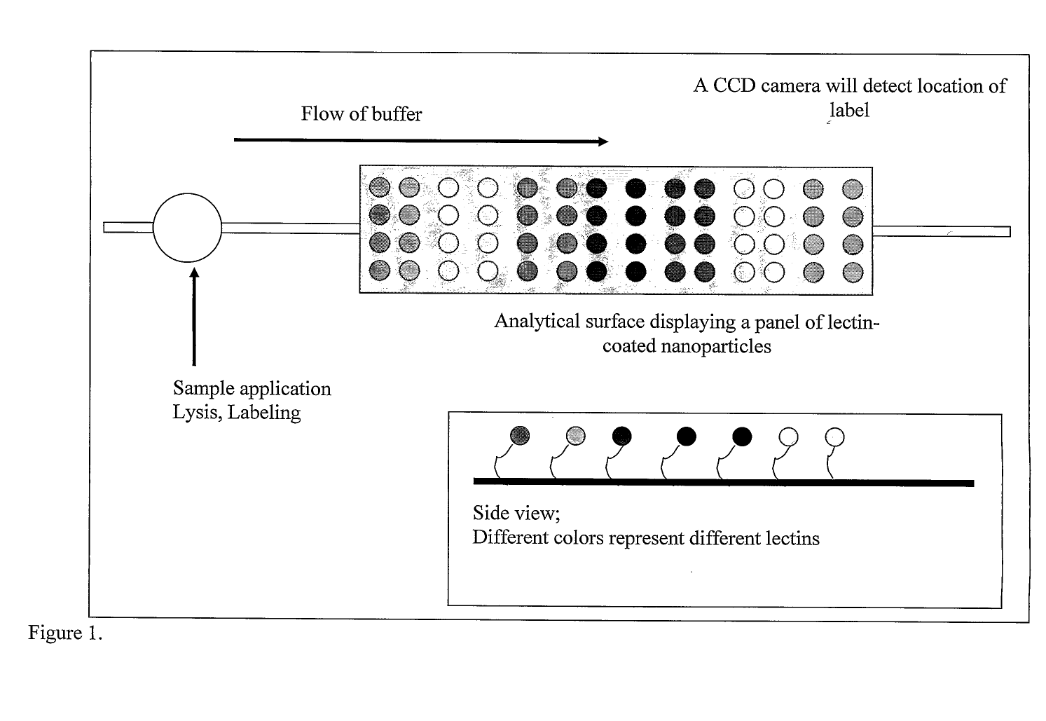

FIG. 2: Schematic view of the ConA-coated particles attached to the analytical surface via oligonucleotide hybridization.

FIG. 3: Fractogram from SdFFF analysis of bare and coated 239 nm PS particles.

FIG. 4: SEM micrographs of the ConA coated particles immobilized on the planar surface. Each spot in the array has a diameter of 300 μm and contains 1.06×10⁶ particles coated with ConA capture-probes.

FIG. 5. Binding of immobilized ConA particles to four different glycoproteins and one unglycosylated protein used as negative control.

### DETAILED DESCRIPTION OF THE INVENTION

In the present invention the mannose-binding lectin ConA has been coupled to polystyrene nanoparticles via a polyethyleneoxide linker which protects the protein conformation and activity and prevents unspecific protein adsorption. The ConA-coated particles are accommodated at different spots on the analytical surface via oligonucleotide linkage. This hybridization method, using complementary oligonucleotides, allows firm attachment of the particles at specific positions. The ConA attached to the particles has retained conformation and activity and binds selectively to a series of different glycoproteins. The results indicate the potential for using the developed multi-lectin nanoparticle arrays in glycoprotein mapping.

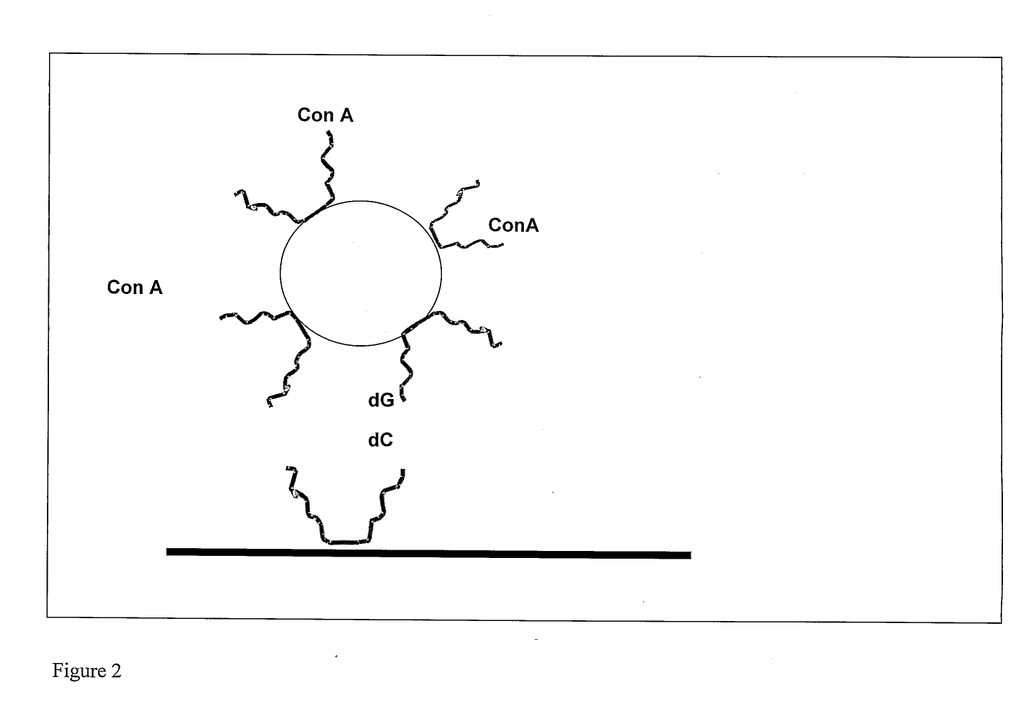

To create a working array system it is important to enable co-immobilization of a number of different capture probes, such as various lectin-coated particles, at specific spots on the analytical read-out surface. This can be done by organized delivery and coupling of the particles to the planar surface via hybridization of complementary oligonucleotide pairs. The use of oligonucleotides gives the possibility to get a nearly infinite number of specific binding sites since the oligonucleotide sequence can be varied in a large number of ways. This has proven to be a robust method allowing firm attachment of the particles to the surface [M Andersson et al]. In the present invention the mannose-binding legume lectin Concanavalin A (ConA) has been used as a model, tethered to the particle surface via Pluronic F108-PDS, The ConA-coated particles have been further functionalized by co-attachment of 15-mers of dG oligonucleotides to the particles, while complementary 15-mers of dC oligonucleotides are immobilized at the planar surface to allow a coupling of the particles as illustrated in FIG. 2. The oligonucleotides are thiolated at the 5′-end which permits covalent attachment to Pluronic F108-PDS coated surfaces. In this study a model experiment has been performed with the ConA-coated particles and a series of four glycoproteins with known carbohydrate-moieties together with one unglycosylated protein, to demonstrate the validity of the developed nanoparticle microarray.

Results

Analysis of Bare and Coated Particles

The development of bioactive nanoparticles necessitates the ability control the exact surface composition of the particles, from precise size of the bare particle to the surface concentration of adsorbed polymer, protein and oligonucleotide locking molecules. Sedimentation Field-Flow Fractionation (SdFFF) is a useful technique for characterization of multilayer composites. This technique allows direct determination of the mass uptake of successively added components on the per particle basis, which is advantageous compared to other techniques for surface concentration measurements. The experimentally determined retention ratio, R, i.e. the ratio of the column void volume, V₀, to the observed retention volume Vᵣ, under a given applied field, is
R=V₀/Vᵣ=6λ[coth(1/2λ)−2λ] (1)

The retention ratio is the basis for determining the λ-value from which the mass of the particles can be calculated according to:
λ=kT/[mₐ(1−ρ_{c}/ρₐ)Gw] (2)
where k is the Boltzmann constant, T is the temperature, mₐ the mass of the particle, ρ_{c} the density of the carrier liquid, ρₐ the density of the particle, w is the channel thickness and G the applied gravitational field. When the mass of the bare particle is known, the mass of the adsorbed or attached layer can be determined as:
λ=kT/[(mₐ(1−ρ_{c}/ρₐ))+(m_{b}(1−ρ_{c}/ρ_{b}))Gw] (3)
where m_{b} is the mass of the adsorbed/attached material and ρ_{b} is its density.

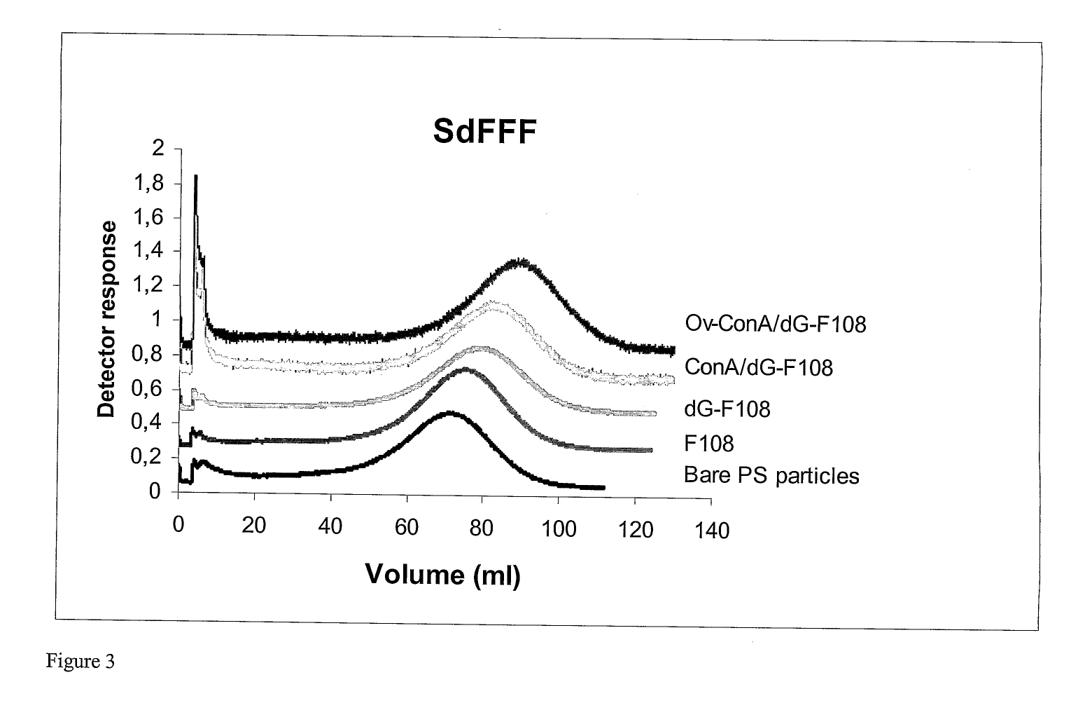

FIG. 3 displays fractograms obtained after injections of bare and coated particles to the SdFFF system. Since the densities of all layers of attached material are exceeding that of the aqueous mobile phase, the uptake of attached material (F108-PDS, dG or ConA) causes significant shifts in elution volumes in the direction of stronger retention. The mass of adsorbed or attached material can easily be calculated according to equation 3 from these shifts in retention. The precision in the surface concentration measurements is 9.4^{x} 10⁻¹⁸ g/PS particle, which in the specific case of our model lectin translates to 55 ConA molecules per bare particle of 239 nm diameter. The uptakes of Pluronic F108-PDS and dG oligonucleotide were also determined with UV spectrophotometry. To examine the biological activity of ConA attached to the particle it was allowed to bind a glycoprotein ligand, in this case Ovalbumin, and the mass increase resulting from this specific binding was determined by SdFFF. Mass determinations for the bare particles and their coating layers are summarized in Table 1. Since ConA has four binding sites per molecule and, in average, 2.8 of these were occupied by ligands (Ovalbumin) it is obvious that the ConA attached to the particles has retained high activity.

TABLE 1
Mass determination of bare particles and their coating layers.

| | | | No of |
|---|---|---|---|
| Bare PS: | Molecular | Surface conc | molecules/ |
| 238.8 ± 1 nm | weight (Da)* | (g/particle) | particle |
| F108-PDS | 14600 | ^{a}3.5 × 10⁻¹⁶ | ^{a}14400 ± 400 |
| | | ^{b}4.6 × 10⁻¹⁶ | ^{b}19100 |
| dG | 5070 | ^{a}5.2 × 10⁻¹⁷ | ^{a}6200 ± 1000 |
| | | ^{b}4.8 × 10⁻¹⁷ | ^{b}5600 |
| ConA | 104000 | 1.2 × 10⁻¹⁶ | 700 ± 55 |
| Ovalbumin | 45000 | 1.5 × 10⁻¹⁶ | 2000 ± 55 |

*Data received from manufacturer
^{a}SdFFF-analysis
^{b}Spectrophotometrically

Immobilization of the Particles on a Read-Out Surface

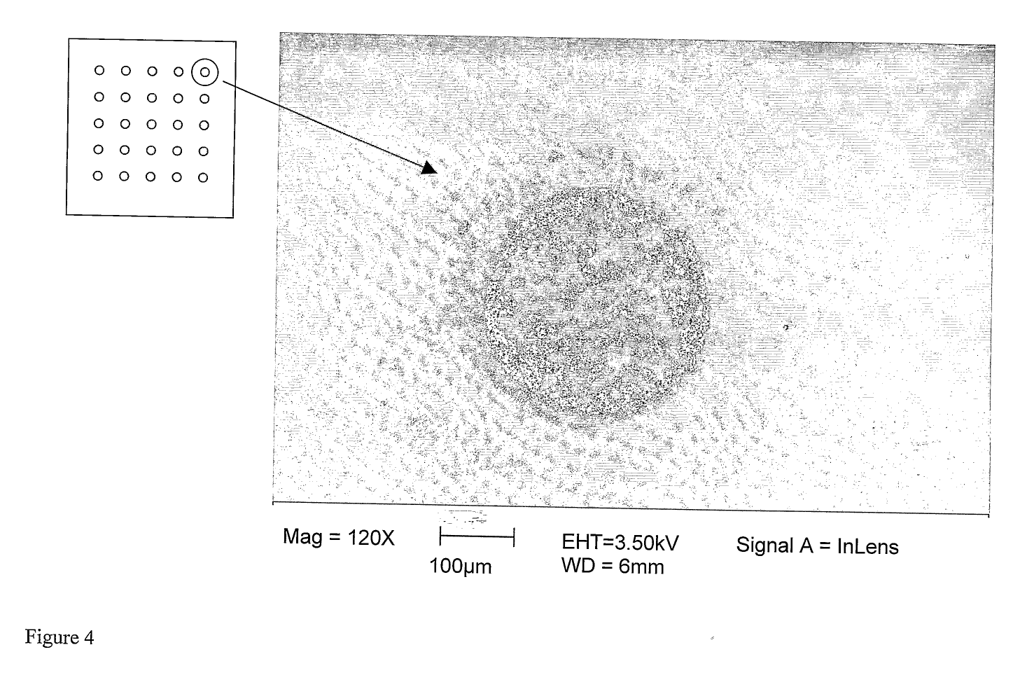

The planar polystyrene read-out surface used has the size of a microscopy slide to which Pluronic F108-PDS has been adsorbed followed by attachment of dC oligonucleotides. The particles in turn were precoated with both complementary dG oligonucleotides and ConA prior to coupling via dC-dG hybridization to the planar surface. A GeSIM Nanoplotter was used to deposit the suspended particles on the surface via 6.4 mL droplets with a 2 mm distance between spots in a 5×5 spot-pattern. The coupling process was allowed to proceed for 20 minutes followed by careful rinsing to remove unbound and loosely bound particles from the substrate surface. The presence of ConA-coated particles attached to the analytical surface was examined by Scanning Electron Microscopy (SEM). When the array is immersed in fluid, the particles are allowed to move with a high degree of freedom around the Pluronic tether. Drying can have a slight effect on their lateral distribution since there is a tendency to minimize the surface. In the system in the dried state, the particles are attracted to each other, rather than appearing as free entities. Therefore, the deposition is performed in a humidified environment. With these precautions, it can be concluded that the particles are reproducibly deposited in spots of equal size, as seen in FIG. 4. The average number of particles accommodated on the surface was counted to 15 per μm₂ and the spot diameter was estimated to 300 μm. This means that there are 1.06×10⁶ particles per dot. Since there are 700 lectin molecules per particle there will be 7.4×10⁸ lectin capture probes per spot of 0.07 mm², each being capable of binding 2.8 ligands.

Glycoprotein Binding to Immobilized ConA

Three well-characterized glycoproteins, Ovalbumin (chicken egg), Fetuin (bovine), Thyroglobulin (porcine) and α-D-Mannosylated-PITC-Albumin (bovine) were selected as model ligands in this study to confirm the activity and selectivity of the immobilized ConA coated particles. Ovalbumin, Fetuin and Thyroglobulin are known to contain several exposed mannose-residues and α-D-Mannosylated-PITC-Albumin is an artificially mannosylated albumin. By contrast Human Serum Albumin (HSA) is not glycosylated and can therefore be used as a negative control. The sample proteins were labelled with Alexa Fluor® 680 fluorescent dye for detection. The degree of labeling (DOL) was estimated spectrophotometrically to be 1.4 for Ovalbumin, 4.2 for Fetuin, 36 for Thyroglobulin, 0.55 for α-D-Mannosylated-PITC-Albumin, and 2.9 for HSA.

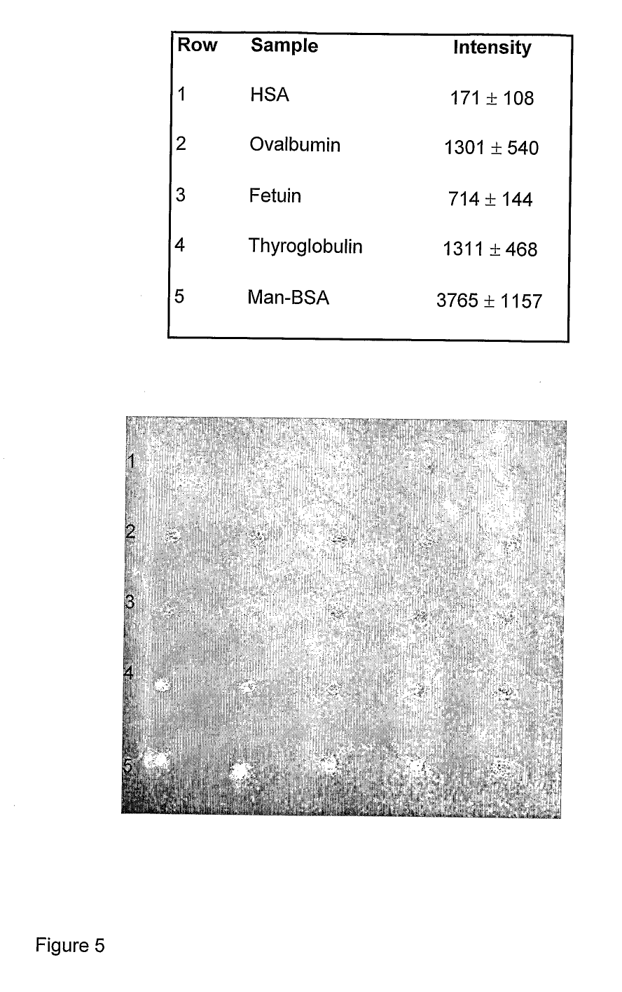

In the present invention, each row of 5 spots with ConA coated particles was exposed to one of the labelled sample proteins (1.3 to 5 ng protein/sample), providing 5 replicate values for each sample. The samples were incubated for 20 minutes followed by repeated washing in 0.05% Tween 20 solution. The slides were placed in a GenePix Scanner for read-out to detect the location of label. The results are presented in FIG. 5 and Table 2. HSA, used as a negative control, gave a very weak signal, seen in row 1, while Ovalbumin, row 2, Thyroglobulin row 4 and α-D-Mannosylated-PITC-Albumin (Man-BSA) row 5 were clearly bound to the ConA as seen from the strong signal. Fetuin, row 3, showed a weaker signal. The ConA coated particles attached to the substrate surface gave rise to very little background fluorescence, which was evaluated from a picture taken before application of labelled sample proteins (not shown).

TABLE 2

| Row | Sample | Intensity |
|---|---|---|
| 1 | HAS | 171 ± 108 |
| 2 | Ovalbumin | 1301 ± 540 |
| 3 | Fetuin | 714 ± 144 |
| 4 | Thyroglobulin | 1311 ± 468 |
| 5 | Man-BSA | 3765 ± 1157 |

FIG. 5. Binding of immobilized ConA particles to four different glycoproteins and one unglycosylated protein used as negative control. The ConA-coated particles spotted out in row 1 have been exposed to HSA, row 2 to Ovalbumin, row 3 to Fetuin, row 4 to Thyroglobulin and row 5 to α-D-Mannosylated-PITC-Albumin. The arrow marks the direction of rows. All five proteins have been labelled with Alexa Fluor®680 dye. The average intensities for the samples are indicated in Table 2.

In the development of lectin-coated nanoparticles it is important to have the exact knowledge of their whole surface chemistry. To provide information of the bare particle as well as their coating layers SdFFF was used. The SdFFF works as a sensitive microbalance and offers a direct and exact determination of the mass increase per particle without any labeling procedures or time consuming washing steps as it leaves the particles well washed and free from loosely adherent material. It allows highly precise surface concentrations of multilayered particles to be determined with a precision in the surface concentration measurement at the attogram level.

Pluronic F108 is known to prevent non-specific protein adsorption to the surface. Nevertheless, minute amounts may still adsorb and cause disturbing background noise in sensitive systems. To further protect the analytical surface from unspecific adsorption all analytes were prepared and washed in 0.05% Tween 20 solution, which decreased traces of non-specifically adsorbed analytes on the surface without release of the polymeric surfactant responsible for the coupling.

It is notable that the particles can be prepared with both proteins and oligonucleotides before coupling to the surface. It could be expected that the large, bulky proteins would shield the small oligonucleotides and thereby complicate the DNA hybridization coupling to the surface. However, as seen from the SEM micrograph the particles were closely packed and evenly distributed over the substrate surface indicating no existing steric hindrance to coupling. From this picture it is also clear that the particles are firmly attached to the surface, since the micrograph is taken after an extensive washing procedure. Particles coated with ConA and dG can be stored in suspension for more than a week and still be functioning if the particle suspension is sonicated prior to application to the substrate surface, as small aggregates tend to form in the suspension during storage. The hybridization technique involving complementary oligonucleotides allows firm and convenient coupling of lectin coated PS particles to a read-out surface. To perform a controlled deposition of large numbers of particles in small spots on the analytical surface, a GeSIM Nanoplotter dispenser was used. This instrument allows small droplets of equal volume to be accommodated, fast and systematically, on the surface. Fast deposition is of utmost importance to avoid drying of the particles on the surface prior to coupling. To further avoid the drying of the surface, the slide is stored in a moisture-chamber during the 15 minutes hybridization reaction.

After deposition the ConA coated particles reproducibly bind a variety of well-characterized mannose-containing glycoproteins, but do not bind the unglycosylated proteins. It was also possible to distinguish between high and low affinity ligands. ConA has high affinity for glycoproteins with a high-mannose content, and preferentially binds mono- and bi-antennary structures, but exhibits less affinity for branched structures of tri- and tetra-antennary complex type. This correlates well with the data in table 2 where a high signal intensity, i.e. strong binding, is shown for Ovalbumin which has one single glycan structure of either high-mannose or hybrid type and Thyroglobulin that contains ca 10% carbohydrate of which about 50% are of bi-antennary high-mannose type. Fetuin with N-linked glycans of the tri-antennary type shows much lower signal intensity and thereby weaker binding. The artificially glycosylated protein, α-D-Mannosylated-PITC-Albumin, has several well-exposed carbohydrate chains constituted of only mannose residues. This sample also shows the strongest binding evaluated from the high intensity. Even if the lectin-carbohydrate binding is relatively weak, binding constants are usually in the 10³-10⁷ M⁻¹ range, the extremely high local concentration of lectin capture probes offered by these particles makes them particularly useful for capture of very dilute samples in complex media.

Although this invention employs ConA as a model lectin to examine the interaction with a series of well characterized glycoproteins, the same strategy will be used with a panel of different lectins accommodated at specific spots on the analytical surface for the analysis of more complex protein samples from e.g. cell lysates. The sample to be analyzed is to be flowed over the lectin panel. Variations in glycosylation will be detected as different patterns of adsorption to the array of nanoparticles with their different loads of sugar-specific lectins. This should enable glycoprotein to be viewed in their entirety without any complex sample preparations and permit large number of samples to be run in parallel. It may therefore have a wide application in both proteomic research and in diagnostics.

Methods and Materials

Polystyrene latex particles with a nominal diameter of 230 nm (10% solids) were purchased from Bangs Laboratories, Inc. (Fishers, Ind., USA). End-group activated Pluronic F108 equipped with pyridyldisulfide groups (Cell-Link™-PDS) were supplied by Allvivo, Inc. (Lake Forest, Calif., USA). Thermo BioSciences GmbH, Ulm, Germany, delivered oligonucleotides of guanine (dG) and cytosine (dC) thiolated at the 5′-end. Dr Bo Ersson kindly supplied Concanavalin A (ConA). Ovalbumin (chicken egg), α-D-Mannosylated-PITC-Albumin (Bovine), Fetuin (Bovine) and Albumin (Human Serum) were purchased from Sigma. Thyroglobulin (Porcine) was from Amersham Biosciences. The buffer used was 10 mM Phosphate containing 0.1 mM CaCl₂ pH 7.4 unless otherwise indicated.

Pluronic F108-PDS Adsorption to Particles and Surfaces

During adsorption a 1-% suspension of PS particles was incubated with 10 g/L F108-PDS in buffer solution for 24 hours at room temperature under constant end-over-end shaking. After adsorption unbound and loosely bound materials were removed by centrifugation in a table-top centrifuge (Eppendorff 5417 C) at 14000 rpm for 20 min followed by removal of supernatant and resuspension of the pellet in buffer. This washing procedure was repeated three times. The supernatant was removed and used for spectrophotometric analysis, while 3 μL was taken out for analysis with SdFFF.

During adsorption the planar surface was placed face down in a solution of Pluronic F108-PDS (10 g/L). The adsorption was allowed to proceed for 24 hours at room temperature followed by washing with buffer solution.

Attachment of Oligonucleotides to the Pluronic F108-Linker

A small portion of thiolated 15-mers of guanine (dG) oligonucleotides (0.5 μL of 100 mM dG) were allowed to bind to some of the PDS-groups on the Pluronic coated PS particles for 1 hour at room temperature. The particles were then washed 3 times by means of centrifugation at 14000 rpm for 20 min. The supernatant was removed after each centrifugation and replaced with buffer solution. A 3 μL sample was taken out for SdFFF analysis.

The Pluronic F108-PDS coated planar PS surface was placed in a 2 nM solution of thiolated 15-mers of cytosine (dC) oligonucleotides and incubated at room temperature for several hours, followed by extensive washing of the surface with buffer solution.

Coupling of ConA to the Particles

In order to bind the ConA to the free pyridyldisulfide groups on the Pluronic F108 adsorbed to the particles, free thiol groups must first be introduced into the ConA molecules. This was done using the heterobifunctional reagent, N-Succinimidyl 3-(2-pyridyldithio) propionate (SPDP). A 10 μl aliquot of 30 mM SPDP-reagent (Sigma) in ethanol was rapidly added to 1 mL of ConA in buffer solution (3.1 mg/mL). The reaction was proceeding for 15 min at room temperature with occasional stirring before excess reagent and low molecular weight products were removed by gelfiltration using a PD-10 column (Amersham Biosciences). The newly introduced 2-pyridyl disulfide was then cleaved off with DTT (Sigma) to give the thiolated ConA. After removal of excess DTT and pyridine-2-thione by gelfiltration with a NAP-10 column (Amersham Biosciences), the thiolated product was immediately transferred to the previously prepared suspension of Pluronic F108-PDS-dG coated particles. The coupling reaction was allowed to proceed for 20 minutes at room temperature. The concentration of pyridyne-2-thione released after cleavage with DTT can be determined from its absorbance at 343 nm. This concentration is equivalent to the concentration of 2-pyridyl disulfide residues in the protein. The degree of substitution with 2-pyridyl disulfide residues was estimated to be 2.2 residues per ConA molecule. A 20 μl sample of the particles was taken out for SdFFF analysis.

Attachment of ConA Coated Particles to the Surface

The suspension of particles coated with ConA and dG was spotted out in 6.4 mL droplets in a 5×5 spots pattern on the analytical surface preadsorbed with Pluronic F108-PDS and saturated with dC, using a GeSIM Nanoplotter dispenser. The complementary dC/dG oligonucleotides were allowed to hybridize in a humid environment at room temperature for 20 minutes followed by repeated rinsing of the surface with buffer solution to wash away all unbound and loosely bound particles.

Scanning Electron Microscopy

The particle distribution was determined by Scanning Electron Microscopy (SEM) when the fluorescence measurements were completed. A FEG-SEM (LEO 1550) was used, and the sample was coated with a thin Au/Pd film prior to imaging at 3.5 kV.

Labeling of Ovalbumin, Fetuin, Thyroglubulin, α-D-Mannosylated-PITC-Albumin and HSA

Ovalbumin, Fetuin, Thyroglobulin, α-D-Mannosylated-PITC-Albumin, and HSA were labelled with Alexa Fluor® 680 Amine-Reactive Probes (Molecular Probes). The protein to be conjugated with the dye was first dissolved at 1-5 mg/mL in 0.1 M sodiumcarbonate buffer pH 8.8. 50 μL of the reactive dye (10 mg/mL in DMSO) was added to the vial containing the protein solution. After a 1-hour incubation the labelled proteins were separated from excess unconjugated dye by gel filtration. The degree of labeling was determined using an UV Spectrophotometer (UV-2101PC, Shimadzu).

Binding of Alexa Fluor® 680-Labelled Ligands to ConA Coated Particles Immobilized at the Analytical Surface

Aliquots of 0.25 μL of the labelled proteins (0.02-0.05 mg/mL) were applied to the spots on the analytical surface to which ConA coated PS particles had been attached. After 15 minutes incubation in a moisture-chamber the surface washed several times with buffer solution. To detect the location of label the surface was put in a GenePix® 4000B Scanner (Axon Instruments, Inc.) for read out using the system's 670-nm laser and 700-nm emission filter. To measure the intensity for the spots, every pixel in the spot is examined and the average intensity is calculated for the whole spot area followed by local background subtraction, using the GenePix® Pro 5.0 Microarray Image analysis software.

Analysis of Particles with Sedimentation Field-Flow Fractionation

The SdFFF system used is a prototype of the commercially available SdFFF system from Postnova (Salt Lake City, USA). The separation takes place in a very thin channel (dimensions 940×20×0.254 mm). The channel is curved to fit inside a rotor basket and positioned 155 mm from the axis of rotation. A PC controls the engine driving the rotor by feedback control. Carrier liquid is fed to the system by a peristaltic pump of type Gilson Minipulse 3, which is controlled by the computer with a predetermined voltage-to-flow relation to keep a constant flow rate. A Sartorious Electronic Precision balance is connected to the computer for continuous measurements of the elution volume (elution weight divided by density of carrier liquid). The signal is monitored by a UV-detector (Pharmacia LKB VWM 2141) held at a fixed wavelength of 254 nm. The output signal from the detector is transferred to the PC. A digital thermometer gives the temperature at the time of start of the experiment. Evaluation of particle size or mass requires the exact knowledge of the densities for the particles and suspension medium. Density measurements were performed using a PAAR density meter; model DMA60+DMA602. The density of the PS particles, Pluronic F108-PDS, dG oligomers and ConA were determined to 1.053, 1.186, 1.65 and 1.35 g/cm³ respectively. The core particles were sized in 0.1% aqueous FL-70 detergent (from Fisher Scientific) while the coated particles were analysed in 0.2 mM NH₄HCO₃. The field strength used was 1400 rpm, the relaxation time was calculated to 11 min and the carrier flow was maintained at 1.5 ml/min throughout each experiment.

### REFERENCES

1. Rudd, P. M., et al. Glycosylation differences between the normal and pathogenic prion protein isoforms. Proc Natl Acad Sci USA. 96, 13044-13049 (1999)
2. Durand, G. & Seta, N. Protein glycosylation and diseases: blood and urinary oligosaccharides as markers for diagnosis and therapeutic monitoring. Clin Chem. 46:6, 795-805 (2000)
3. Rudd, P. M., Elliott, T., Cresswell, P., Wilson, I. A. & Dwek, R. A. Glycosylation and the immune system. Science. 291, 2370-2376 (2001)
4. Jaeken, J. Komrower lecture. Congenital disorders of glycosylation (CDG): J Inherit Metab Dis. 26 99-118 (2003)
5. Turner, G. A. N-glycosylation of serum proteins in disease and its investigation using lectins. Clin Chim Acta. 208, 149-171 (1992)
6. Lis, H. & Sharon N. Lectins: Carbohydrate-specific proteins that mediate cellular recognition. Chem Rev. 98, 637-674 (1998)
7. Nilsson, C. L. Lectins: proteins that interpret the sugar code. Anal Chem. 15, 349-353 (2003)
8. Mitchell, P. A perspective on protein microarrays. Nat Biotechnol., 20, 225-229 (2002)
9. Gershon, D. Probing the proteome. Nature, 424.581-587 (2003)
10. Fromell, K., Huang, S. C. & Caldwell, K. D. Scanning calorimetry in probing the structural stability of proteins at interfaces. Biopolymers at Interfaces, M. Malmsten, 2^{nd} ed., Marcel Dekker, New York, 517-538 (2003)
11. Yan, G. Y., Li, J.-T., Huang, S.-C. & Caldwell, K. D. Calorimetric Observations of Protein Conformation at Solid/Liquid Interfaces. Advances in Chemistry 602: Proteins at Interfaces II, T. Horbett and J. Brash, Eds., ACS Press, Washington, 256-268 (1995)
12. Norde, W. & Zoungrana, T. Surface-induced changes in the structure and activity of enzymes physically immobilized at solid/liquid interfaces. Biotechnol Appl Biochem. 28, 133-143 (1998).
13. Li, J.-T., Carlsson, J., Lin, J. N. & Caldwell, K. D. Chemical modification of surface active poly(ethylene oxide)-poly (propylene oxide) triblock copolymers. Bioconjug Chem. 7, 592-599 (1996).
14. Neff, J. A., Tresco, P. A. & Caldwell, K. D. Surface modification for controlled studies of cell-ligand interactions. Biomaterials. 20, 2377-2393 (1999)
15. Giddings, J. C. Field-flow fractionation: Analysis of macromolecular, colloidal, and particulate materials. Science. 260, 1456-1465 (1993)
16. Li, J.-T & Caldwell, K. D. Sedimentation field-flow fractionation in the determination of surface concentration of adsorbed materials. Langmuir. 7, 2034-2039 (1991)
17. Li, J.-T., Caldwell, K. D. & Rapoport, N. Surface properties of Pluronic coated polymeric colloids. Langmuir. 10, 4475-(1994)
18. Caldwell, K. D., Li, J.-T., Li, J.-M. & Dalgleish, D. G. Adsorption behavior of milk proteins on polystyrene latex: A study based on sedimentation field-flow fractionation and light scattering. J Chomatogr. 604, 63-71 (1992)

1. A device for protein analysis, comprising an array of particulate nanoparticles, wherein said nanoparticles are bound at specific spots on a planar read-out surface and are provided with a plurality of capture probes for capturing proteins.

2. A device according to claim 1, wherein the proteins are glycoproteins and the capture probes are lectins.

3. A device according to claim 1, wherein the array of nanoparticles is divided into several sections and each section having different capture probes attached to the nanoparticles.

4. A device according to claim 1, wherein the nanoparticles are bound to the solid phase by oligomers on the nanoparticles and complementary oligomers on the read-out surface.

5. A device according to claim 1, wherein the capture probes are attached to the nanoparticles via a polyethyleneoxide linker.

6. A device according to claim 2, wherein the lectins are selected from the followings groups:
mannose-specific lectins: Concanavalin A/ConA, type 1 fimbriae, favin, GNL, LOL, LCL, MBP-A, PSL; N-acetylglucoseamine-specific lectins: GSII, WGA; galactose/N-acetylgalactoseamine-specific lectins: jacalin, DBL, ECorL, LBA, MLL, PNA, RCAII, SBA; fucose-specific lectins: LTA, UEA I; sialic acid-specific lectins: lectin from Sambucus nigra (elderberry).

7. A method for protein analysis, comprising the following steps:
a) application of a sample to the device according to claim 1,
b) binding of proteins in the sample to the capture probes,
c) incubation,
d) washing off excess sample, and
e) detection of possibly bound proteins.

8. A method according to claim 7, wherein the capture probes are lectins and the proteins are qlycoproteins.

9. A device according to claim 2, wherein the array of nanoparticles is divided into several sections and each section having different capture probes attached to the nanoparticles.

10. A device according to claim 2, wherein the nanoparticles are bound to the solid phase by oligomers on the nanoparticles and complementary oligomers on the read-out surface.

11. A device according to claim 3, wherein the nanoparticles are bound to the solid phase by oligomers on the nanoparticles and complementary oligomers on the read-out surface.

12. A device according to claim 2, wherein the capture probes are attached to the nanoparticles via a polyethyleneoxide linker.

13. A device according to claim 3, wherein the capture probes are attached to the nanoparticles via a polyethyleneoxide linker.

14. A device according to claim 4, wherein the capture probes are attached to the nanoparticles via a polyethyleneoxide linker.
